(19) United States
(12) Patent Application Publication
(10) Pub. No.: US 2020/0138880 A1
(43) Pub. Date: May 7, 2020

(54) BACTERIA-BASED GEL COMPOSITIONS FOR TOPICAL APPLICATIONS AND USES THEREOF

(71) Applicants: Vera Mogna, Novara (IT); PROBIOTICAL S.P.A., Novara (IT)

(72) Inventor: Giovanni Mogna, Novara (IT)

(21) Appl. No.: 16/305,029

(22) PCT Filed: May 31, 2017
(86) PCT No.: PCT/IB2017/053206

§ 371 Date: Nov. 27, 2018

(30) Foreign Application Priority Data
Jun. 1, 2016 (IT) 102016000057122

(51) Int. Cl. A61K 35/747; A61K 35/745; A61K 9/00; A61K 9/06; A61K 47/10; A61K 31/4045

(52) U.S. Cl. CPC A61K 35/747; A61K 35/745; A61K 31/4045; A61K 9/06; A61K 47/10; A61K 9/0014

(57) ABSTRACT

The present invention relates to a composition for use as a medicament, in the form of a gel with characteristics of a reversible thermogel, based on one or more poloxamers, water and optionally excipients, and an effective amount of an active substance. Furthermore, the present invention relates to a process for the preparation of said composition and uses thereof.

The present invention relates to a composition for use as a medicament, in the form of a gel with characteristics of a reversible thermogel, based on one or more poloxamers, water and optionally excipients, and an effective amount of an active substance. Furthermore, the present invention relates to a process for the preparation of said composition and uses thereof.

In the market, there exist various forms of administration of active ingredients, e.g. microorganisms such as bacteria, at least one flavonoid such as rutin, oxerutin, diosmin, hesperidin and troxerutin, chemotherapeutic agents and/or arctigenin, arctin, berberine, berbamine, sanguinarine and chelerythrine, or rutoxides, as such or in the form of plant extracts containing said compounds, or melatonin or derivatives thereof. The topical forms of administration can be patches, creams, gels or suspensions. An example of a gel composition is described in EP2520279 A1.

However, there continues to be a felt need to be able to have a form of administration for topical use for transdermal application that is economical and easy to prepare, stable, efficacious in dosing the effective amount of active substance contained therein, and easily absorbable at the level of the cutis, dermis and epidermis without leaving residues or streaks.

The Applicant has developed a composition in gel form for topical use which provides an adequate and advantageous response to the above-mentioned needs.

The present invention relates to a composition in the form of gel for topical use, having the features as claimed in the accompanying claims.

The present invention relates to a process for the preparation of said composition, having the features as claimed in the accompanying claims.

The present invention relates to a composition for use as a medicament in gel form or topical use, having the features as claimed in the accompanying claims.

The preferred embodiments of the present invention set forth in the description that follows are illustrated solely by way of example and in no way limit the broad scope of application of the present invention, which will appear clear to the person skilled in the art.

In the context of the present invention, composition(s) means a pharmaceutical composition, a composition for medical devices, a composition for dietary supplements or a food composition.

Advantageously, the composition of the present invention is capable of releasing the active substance or active ingredient over time, in a constant, gradual and lasting manner as a controlled transdermal release, in such a way as to prolong its activity and therapeutic effectiveness over time.

Advantageously, the composition of the present invention is a gel in the form of a reversible therrnogel thanks to the presence of a vehicle or carrier which contains specific selected polymers of the poloxamer type.

Advantageously, the composition of the present invention is for topical use for transdermal and/or transmucosal application, said composition is furthermore economical and easy to prepare, stable, efficacious in dosing the effective amount of active substance contained therein over time, and easily absorbable at the level of the cutis, dermis and epidermis without leaving residues or streaks.

The present invention relates to a composition (abbreviated as CMP) in the form of a gel for topical use. Said composition comprises:
an effective amount of an active substance selected from the group comprising or, alternatively, consisting of:
   (i) at least one microorganism selected from the group comprising or, alternatively, consisting of: live lactic bacteria, live bifidobacteria, biologically active bacteria or active bacterial components, extracts or cell components, bacterial enzymes, tyndallized bacteria, lysed bacteria, sonicated bacteria and peptidoglycans; and
   (ii) melatonin and/or the natural and/or synthetic derivatives thereof;
   preferably (iii) at least one compound selected from the group comprising or, alternatively, consisting of a flavonoid such as rutin, rutoxides, arbutin, oxerutin, diosmin, hesperidin and troxerutin, chemotherapeutic agents, arctigenin, arctin, berberine, berbamine, sanguinarine and chelerythrine, as such or in the form of plant extracts containing said compounds, on their own or in combination with one or more chemotherapeutic agents or anti-tumour drugs and a vehicle or carrier, wherein said composition comprises or, alternatively, consists of an effective amount of a microorganism (i) selected from the group comprising or, alternatively, consisting of the microorganisms in Table 1; preferably said at least one microorganism is selected from the group comprising or, alternatively, consisting of the strains: Lactobacillus salivarius (LS01) DSM 22775, deposited on 23 Jul. 2009; Bifidobacterium breve (BR03) DSM 16604, deposited on 20 Jul. 2004; Lactobacillus pentosus (LPS01) DSM 21980, deposited on 14 Nov. 2008; Streptococcus thermophilus (FP4) DSM 18616, deposited on 13 Sep. 2006; Lactobacillus casei ssp. rhamnosus (LR04) DSM 16605, deposited on 20 Jul. 2004; and Lactobacillus acidophilus (LA02) DSM 21717, deposited on 6 Aug. 2008; even more preferably the strain is Lactobacillus salivarius (LS01) DSM 22775, and/or Bifidobacterium breve (BR03) DSM 16604, and/or L. pentosus (LPS01) DSM 21980, and wherein said composition is for topical transdermal use in the preventive and/or curative treatment of pathologies, disorders or diseases associated with/deriving from alterations of the immune system selected from the group comprising allergies, atopy, allergic rhinitis, food hypersensitivity, dermatitis, atopic dermatitis, eczema, psoriasis, asthma and immunodeficiencies. Said vehicle or carrier comprises water, preferably purified water, until reaching 100% by weight of the composition, and a thickening viscous matrix. Said thickening viscous matrix comprises or, alternatively, consists of at least one poloxamer or a mixture of poloxamers and, optionally, additives and/or excipients and/or adjuvants. Said additives and/or excipients and/or adjuvants are selected from among those capable of promoting the formation and stabilisation of the gel (reversible thermogel) and are selected from the group comprising or, alternatively, consisting of acid salts, sodium sorbate, potassium sorbate, sodium benzoate, potassium benzoate, glycols, ethylene glycol and propylene glycol.

In a preferred embodiment (CMP1), the composition of the present invention (CMP) comprises a poloxamer which is selected from the group comprising or, alternatively, consisting of Poloxamer 124 CAS N. 9003-11-6 with an average molecular weight of 2090-2360, Poloxamer 188 (Lutrol F66) CAS No. 9003-11-6 with an average molecular weight of 7680-9510, Poloxamer 237 CAS N, 9003-11-6 with an average molecular weight of 6840-8830, Poloxamer 338 CAS N 9003-11-6 with an average molecular weight of 12700-17400, Poloxamer 407 (Lutrol® F127 Prill) CAS No. 9003-11-6 with an average molecular weight of 9840-14600, or mixtures thereof; preferably said Poloxamer 188 is present in an amount comprised from 0.1% to 10% by weight, out of 100 g of composition, preferably in an amount comprised from 0.5% to 5% by weight, for example from 1% to 3% by weight; and/or preferably said Poloxamer 407 is present in an amount comprised from 1% to 40% by weight, out of 100 g of composition, preferably in an amount comprised from 5% to 30% by weight, for example from 10% to 20% by weight (composition CMP1). Advantageously, the composition CMP1 contains Poloxamer 188 (Lutrol F66) CAS No. 9003-11-6 with an average molecular weight of 7680-9510 and/or Poloxamer 407 (Lutrol F127 Prill) CAS No. 900-11-6 with an average molecular weight of 984014600.

In another preferred embodiment (CMP4), the composition CMP or CMP1 comprises said microorganisms at a concentration comprised from 1×10⁶ CFU/g to 1×10¹² CFU/g of composition, preferably from 1×10⁷ CFU/g to 1×10¹¹ CFU/g of composition, even more preferably from 1×10⁸ CFU/g to 1×10¹⁰ CFU/g of composition, for example 1×10⁹ CFU/g of composition, and wherein:
said microorganisms are present in said composition in an amount by weight comprised from 0.1% to 5%, preferably in an amount by weight comprised from 0.5% to 3%, even more preferably in an amount by weight comprised from 1% to 2%, relative to the total weight of the composition (CMP4).

In another preferred embodiment (CMP5), the composition CMP, CMP1 or 4 comprises an effective amount of an active substance selected from:

(iii) at least one compound selected from the group comprising or, alternatively, consisting of a flavonoid such as rutin, rutoxides, arbutin, oxerutin, diosmin, hesperidin and troxerutin, chemotherapeutic agents, arctigenin, arctin, berberine, berbamine, sanguinarine and chelerythrine, as such or in the form of plant extracts containing said compounds, on their own or in combination with one or more chemotherapeutic agents or anti-tumour drugs; and
(ii) melatonin and/or the natural and/or synthetic derivatives thereof;
said active substance being present in said composition in an amount by weight comprised from 0.1% to 10% by weight, preferably in an amount comprised from 0.5% to 5% by weight, even more preferably from 1% to 3% by weight, relative to the total weight of the composition (CMP5).

In another embodiment (CMP6), the composition as described above is for use:
in the treatment of pathologies, disorders or diseases due to the pathological malfunctioning of physiological functions connected with circadian rhythms;
in the treatment of pathologies, disorders or diseases due to an abnormal or excessively high or excessively low blood pressure;
in the treatment of pathologies, disorders or diseases due to an excess of free radicals and oxidative stress;
in the treatment of pathologies, disorders or diseases due to an imbalance of the immune system;
in the treatment of pathologies, disorders or diseases due to inflammatory states;
in the treatment of pathologies, disorders or diseases due to acquired immunodeficiency;
in the treatment of pathologies, disorders or diseases due to viral infections and bacterial infections;
in a treatment as a support or adjuvant to chemotherapy;
in the treatment of cancer with chemotherapeutic agents;
in the treatment of sleep disorders in order to favour, in the treated subjects, the quality of sleep and regularity of sleep, with particular reference to REM sleep;
in the treatment of pathologies, disorders or diseases of the eyes, of the retina, of the iris, of the optic nerve, retinal detachment and macular degeneration;
in the treatment of vision disorders, in particular in maculopathy of the retina, via external applications in the vicinity of the eye and the temple;
in the treatment of pathologies, disorders or diseases due to autism and Down's syndrome, in particular in children and adolescents, by favouring balance and control also of states of anxiety;
in the treatment of pathologies, disorders or diseases due to senile dementia or Alzheimer's disease;
in the treatment of pathologies, disorders or diseases due to Parkinson's disease or neurological dysfunctions, by improving sleep and tremors;
in the treatment of pathologies, disorders or diseases due to pulmonary cystic fibrosis in children, by performing an anti-inflammatory activity;
in the treatment of prostate tumours, by preventing the propagation thereof;
in the treatment of tumours, as an anti-inflammatory, anti-tumour adjuvant;
in the treatment of pathologies, disorders or diseases due to heart attack, myocardial infarction, cardiopathies, cardiac or coronary failure or myocardial insufficiency.

The present invention relates to a process for the preparation of a composition in accordance with the compositions CMP or CMP1 or 4 or 5 or 6, said process comprising the following steps:
mixing in water, under continuous stirring, the various components, added in succession one after the other or separately premixed and added together, using a mixer provided with a temperature control and adjustment means and a stirring means, wherein the temperature is set at values lower than room temperature, comprised from 2° C. to 16° C., preferably from 4° C. to 10° C., for example from 6° C. to 8° C., for a time comprised from 1 minute to 15 minutes so as to obtain a homogeneous mixture;
maintaining said homogeneous mixture, preferably under continuous stirring, at a temperature comprised from 2° C. to 10° C., preferably at a temperature comprised from 4° C. to 8° C., for example from 5° C. to 7° C., for a time comprised from 1 minute to 60 minutes, preferably from 5 minutes to 40 minutes, even more preferably from 10 minutes to 20 minutes, so as to obtain said composition in a liquid state;
packaging said composition in sealed containers.

The vehicle present in the composition of the present invention comprises water and a thickening viscous matrix comprising at least one poloxamer or a mixture of at least due poloxamers.

In the context of the present invention, the term “reversible thermogel” refers to a gel obtained from aqueous copolymer solutions, wherein the copolymer is a poloxamer, capable of gelling in a reversible manner according to the temperature.

Typically, the composition, i.e. the reversible thermogel, of the present invention is liquid at temperatures below room temperature and becomes gelatinous at temperatures close to human body temperature. For example, said composition is liquid at temperatures approximately comprised from 3° C. to 15° C.; in particular, at about 5° C. Said composition in turn gels, that is, becomes a gel, starting from about 20° C. until about 37°; in particular it gels between about 20° and 25° C., preferably, between about 21° and 23° C.

In the context of the present invention, the active agent, for example the melatonin, is present in the composition of the invention in an amount comprised from 0.1% to 3% by weight, out of 100 g of composition; preferably, comprised from 0.5% to 2% by weight, out of 100 g of composition: more preferably, about 1% by weight, out of 100 g of composition; even more preferably, 1% by weight, out of 100 g of composition. The melatonin can be added into the composition as such, in powder, with a purity comprised from 96% to 99.9%; in particular, with a purity comprised from 97% to 99%; for example, with a purity of the 98%. Alternatively, the melatonin can be added into the composition also in the form of microparticles, for example of a size comprised from 50 to 100 μm.

In the context of the present invention, the poloxamers are a series of block copolymers of ethylene oxide and propylene oxide in accordance with the following structural formula;

HO(C₂H₄O)₈(C₃H₆O)_{b}(C₂H₄O)ₐH,

where a and b are whole numbers expressing the number of the oxyethylene and oxypropylene residues present in the molecule. Poloxamers are non-ionic copolymers of polyoxyethylene-polyoxypropylene used primarily in pharmaceutical formulations as emulsifying, solubilising or welling agents.

The poloxamers of the present invention are capable of thickening the aqueous solutions and giving rise to reversible thermogels possessing rheological properties that vary according to their concentration and molecular weight. A large number of poloxamers (whose molecular weight varies widely according to the values of a and b) can be used for the purposes of the present invention. Among them, the five poloxamers (characterised by the codes 124, 188, 237, 338, 407, respectively) included in the US Pharmacopeia USP NF XVII, in the chapter headed “Poloxamer”, whose contents are incorporated herein in their entirety, have proven to be preferable. Among these, the poloxamers are preferably selected from the group consisting of Poloxamer 188 (Lutrol® F 68, BASF SE, Ludwigshafen, DE), CAS 9003-11-6; Poloxamer 407 (Lutrol® F 127, BASF SE, Ludwigshafen, DE), CAS 900-11-6; or mixtures thereof. In a preferred embodiment of the invention, a mixture of Poloxamer 188 and Poloxamer 407 is used.

In the context of the present invention, the at least one poloxamer or mixture of poloxamers is present in a total amount comprised from 1% to 35% by weight, out of 100 g of composition.

In a preferred embodiment of the invention,

the Poloxamer 188 is present in an amount comprised from 1% to 5% by weight, out of 100 g of composition; preferably, comprised from 1% to 3% by weight, out of 100 g of composition; more preferably, comprised from 1% to 2% by weight, out of 100 g of composition; even more preferably, of 1% by weight, out of 100 g of composition; and
the Poloxamer 407 is present in an amount comprised from 15% to 30% by weight, out of 100 g of composition; preferably, comprised from 18% to 25% by weight, out of 100 g of composition; more preferably, comprised from 20% to 23% by weight, out of 100 g of composition; even more preferably, of 21% by weight, out of 100 g of composition.

In the context of the present invention, the water is preferably purified water and is present in an amount such as to reach a balance at 100% by weight of the composition.

The composition of the present invention further comprises known additives/excipients/adjuvants commonly used in the pharmaceutical formulation technique; in particular, said additives/excipients/adjuvants are selected from among those particularly advantageous for the formation and the stabilisation of the reversible thermogel of the invention. In a preferred embodiment of the invention, said additives/excipients/adjuvants are selected from the group consisting of acid salts, such as sodium sorbate, potassium sorbate, sodium benzoate, potassium benzoate; and glycols, such as ethylene glycol and propylene glycol.

In a particularly preferred embodiment, said additives/excipients/adjuvants consist of potassium sorbate, sodium benzoate and propylene glycol, in a total amount comprised from 1 to 5% by weight, out of 100 g of composition.

The pharmaceutical composition for topical use in the form of a reversible thermogel of the present invention can be prepared by adopting well-known apparatus and processing conditions commonly used in the industry for the preparation of a reversible thermogel.

Essentially, the various ingredients making up the desired composition, added in succession or pre-mixed, undergo cold mixing using a suitable mixer provided with a refrigeration temperature control and adjustment means and a stirring means. The mixing temperature is set on values below room temperature, preferably between 3 and 8° C., and the mixture of components is kept under cold stirring for an amount of time sufficient to obtain a completely homogeneous mixture which, in an amount of time that will vary depending on the type of poloxamers used, will transform, still cold, into a completely liquid solution.

The product thus obtained is then packaged in suitable containers, e.g. sealed tubes, and subsequently, on reaching room temperature, will transform into a gel, in particular at the moment when it is applied on the skin.

In one embodiment (FR1), the present invention relates to a composition in the form of a gel for topical use comprising:
an effective amount of an active substance selected from the group comprising or, alternatively, consisting of:
   (i) at least one microorganism selected from the group comprising or, alternatively, consisting of: live lactic bacteria, live bifidobacteria, biologically active bacteria or active bacterial components, cellular extracts or components, bacterial enzymes, tyndallized bacteria, lysed bacteria, sonicated bacteria and peptidoglycans; and/or
   (ii) at least one compound selected from the group comprising or, alternatively, consisting of a flavonoid such as rutin, rutoxides, arbutin, oxerutin, diosmin, hesperidin and troxerutin, chemotherapeutic agents, arctigenin, arctin, berberine, berbamine, sanguinarine and chelerythrine, as such or in the form of plant extracts containing said compounds, on their own or in combination with one or more chemotherapeutic agents or anti-tumour drugs; and/or
   (iii) melatonin and/or the natural and/or synthetic derivatives thereof; and
a vehicle or carrier comprising water, a thickening viscous matrix comprising or, alternatively, consisting of at least one poloxamer or a mixture of poloxamers and, optionally, additives and/or excipients and/or adjuvants selected from among those capable of promoting the formation and stabilisation of the gel, selected from the group comprising or, alternatively, consisting of acid salts, sodium sorbate, potassium sorbate, sodium benzoate, potassium benzoate, glycols, ethylene glycol and propylene glycol; said composition being for use as a medicament.

In a preferred embodiment (FR2), the present invention relates to the composition for use according to FR1, wherein said poloxamer is selected from the group comprising or, alternatively, consisting of Poloxamer 124 with an average molecular weight of 2090-2360, Poloxamer 188 (Lutrol F66) CAS No. 9003-11-6 with an average molecular weight of 7680-9510, Poloxamer 237 with an average molecular weight of 6840-8830, Poloxamer 338 with an average molecular weight of 12700-17400, Poloxamer 407 (Lutrol F127 Prill) CAS No. 900-11-6, with an average molecular weight of 984014600, or mixtures thereof; preferably said Poloxamer 188 is present in an amount comprised from 0.1% to 10% by weight, out of 100g of composition, preferably in an amount comprised from 0.5% to 5% by weight, for example from 1% to 3% by weight; and/or preferably said Poloxamer 407 is present in an amount comprised from 1% to 40% by weight, out of 100g of composition, preferably in an amount comprised from 5% to 30% by weight, for example from 10% to 20% by weight.

In a preferred embodiment (FR3), the present invention relates to the composition for use according to FR1, wherein:
said composition comprises or, alternatively, consists of an effective amount of an active substance selected from at least one microorganism (i) and, wherein:
said composition is for topical transdermal use in the preventive and/or curative treatment of pathologies, disorders or diseases associated with/deriving from alterations of the immune system selected from the group comprising allergies, atopy, allergic rhinitis, food hypersensitivity, dermatitis, atopic dermatitis, eczema, psoriasis, asthma and immunodeficiencies.

In a preferred embodiment (FR4), the present invention relates to the composition for use according to one of FR1-FR3, wherein said at least one microorganism is selected from the group comprising or, alternatively, consisting of the microorganisms in Table 1; preferably said at least one microorganism is selected from the group comprising or, alternatively, consisting of the strains: Lactobacillus salivarius (LS01) DSM 22775, deposited on 23 Jul 2009; Bifidobacterium breve (BR03) DSM 16604, deposited on 20.072004; Lactobacillus pentosus (LPS01) DSM 21980, deposited on 14 Nov. 2008; Streptococcus thermophilus (FP4) DSM 18616, deposited on 13 Sep. 2006; Lactobacillus casei ssp. rhamnosus (LR04) DSM 16605, deposited on 20 Jul. 2004; and Lactobacillus acidophilus (LA02) DSM 21717, deposited on 6 Aug. 2008; even more preferably the strain is Lactobacillus salivarius (LS01) DSM 22775, and/or Bifidobacterium breve (BR03) DSM 16604, and/or L. pentosus (LPS01) DSM 21980.

In a preferred embodiment (FR5), the present invention relates to the composition for use according to FR4, wherein said microorganisms are present at a concentration comprised from 1×10⁶ CFU/g to 1×10¹² CFU/g of composition, preferably from 1×10⁷ CFU/g to 1×10¹¹ CFU/g of composition, even more preferably from 1×10⁸ CFU/g to 1×10¹⁰ CFU/g of composition, for example 1×10⁹ CFU/g of composition, and wherein:
said microorganisms are present in said composition in an amount by weight comprised from 0.1% to 5%, preferably in an amount by weight comprised from 0.5% to 3%, even more preferably in an amount by weight comprised from 1% to 2%, relative to the total weight of composition.

In a preferred embodiment (FR6), the present invention relates to the composition for use according to FR1 or the FR2, wherein:
said composition comprises or, alternatively, consists of an effective amount of an active substance selected from:
   (ii) at least one compound selected from the group comprising or, alternatively, consisting of a flavonoid such as rutin, rutoxides, arbutin, oxerutin, diosmin, hesperidin and troxerutin, chemotherapeutic agents, arctigenin, arctin, berberine, berbamine, sanguinarine and chelerythrine, as such or in the form of plant extracts containing said compounds, on their own or in combination with one or more chemotherapeutic agents or anti-tumour drugs; and/or
   (iii) melatonin and/or the natural and/or synthetic derivatives thereof;
   said active substance being present in said composition in an amount by weight comprised from 0.1% to 10% by weight, preferably in an amount comprised from 0.5% to 5% by weight, even more preferably from 1% to 3% by weight, relative to the total weight of the composition.

In a preferred embodiment (FR7), the present invention relates to the composition for use according to one of FR1-FR6, for use:
in the treatment of pathologies, disorders or diseases due to the pathological malfunctioning of physiological functions connected with circadian rhythms;
in the treatment of pathologies, disorders or diseases due to an abnormal or excessively high or excessively low blood pressure;
in the treatment of pathologies, disorders or diseases due to an excess of free radicals and oxidative stress;
in the treatment of pathologies, disorders or diseases due to an imbalance of the immune system;
in the treatment of pathologies, disorders or diseases due to inflammatory states;
in the treatment of pathologies, disorders or diseases due to acquired immunodeficiency;
in the treatment of pathologies, disorders or diseases due to viral infections and bacterial infections;
in a treatment as a support or adjuvant to chemotherapy;
in the treatment of cancer with chemotherapeutic agents;
in the treatment of sleep disorders in order to favour, in the treated subjects, the quality of sleep and regularity of sleep, with particular reference to REM sleep;
in the treatment of pathologies, disorders or diseases of the eyes, of the retina, of the iris, of the optic nerve, retinal detachment and macular degeneration;
in the treatment of vision disorders, in particular in maculopathy of the retina, via external applications in the vicinity of the eye and the temple;
in the treatment of pathologies, disorders or diseases due to autism and Down's syndrome, in particular in children and adolescents, by favouring balance and control also of states of anxiety;
in the treatment of pathologies, disorders or diseases due to senile dementia or Alzheimer's disease;
in the treatment of pathologies, disorders or diseases due to Parkinson's disease or neurological dysfunctions, by improving sleep and tremors;
in the treatment of pathologies, disorders or diseases due to pulmonary cystic fibrosis in children, by performing an anti-inflammatory activity;
in the treatment of prostate tumours, by preventing the propagation thereof;
in the treatment of tumours, as an anti-inflammatory, anti-tumour adjuvant;
in the treatment of pathologies, disorders or diseases due to heart attack, myocardial infarction, cardiopathies, cardiac or coronary failure or myocardial insufficiency,

In a preferred embodiment (FR8), the present invention relates to a process for the preparation of a composition in accordance with any one of FR1-FR7, said process comprising the following steps:
mixing in water, under continuous stirring, the various components, added in succession one after the other or separately premixed and added together, using a mixer provided with a temperature control and adjustment means and a stirring means, wherein the temperature is set at values lower than room temperature, comprised from 2° C. to 16° C., preferably from 4° C. to 10° C., for example from 6° C. to 8° C., for a time comprised from 1 minute to 15 minutes so as to obtain a homogeneous mixture;
maintaining said homogeneous mixture, preferably under continuous stirring, at a temperature comprised from 2° C. to 10° C., preferably at a temperature comprised from 4° C. to 8° C., for example from 5° C. to 7° C., for a time comprised from 1 minute to 60 minutes, preferably from 5 minutes to 40 minutes, even more preferably from 10 minutes to 20 minutes, so as to obtain said composition in a liquid state;
packaging said composition in sealed containers.

Solely by way of example, which does not limit the various possibilities of production, an example of preparation of a reversible thermogel/composition of the present invention is described here below.

### EXAMPLE 1—PREPARATION OF A LOT OF A COMPOSITION (IN THE FORM OF A REVERSIBLE THERMOGEL) OF THE PRESENT INVENTION

| Component | weight (g) | % |
|---|---|---|
| Mixture of bacteria | 2.00 | 1.00 |
| (1) or (2) or (3) or (4) or (5) | | |
| Poloxamer 188 | 4.00 | 2.00 |
| Poloxamer 407 | 40.00 | 20.00 |
| K sorbate | 0.40 | 0.20 |
| Na benzoate | 1.00 | 0.50 |
| Propylene glycol | 2.00 | 1.00 |
| Purified water | 150.60 | 75.30 |
| TOTAL | 200.00 | 100.00 |

Mixture (1) consisting of only Lactobacillus salivarius (LS01) DSM 22775, or only Bifidobacterium breve (BR03) DSM 16604, or only L. pentosus (LPS01) OSM 21980.

Mixture (2) consisting of Lactobacillus salivarius (LS01) DSM 22775, Bifidobacterium breve (BR03) DSM 16604 and L. pentosus (LPS01) DSM 21980, in a ratio by weight of 1:1:1.

Mixture (3) consisting of Lactobacillus salivarius (LS01) DSM 22775 and Bifidobacterium breve (BR03) DSM 16604 in a ratio by weight of 1:1.

Mixture (4) consisting of Lactobacillus salivarius (LS01) DSM 22775 and L. pentosus (LPS01) OSM 21980, in a ratio by weight of 1:1.

Mixture (5) consisting of Bifidobacterium breve (BR03) DSM 16604 and L. pentosus (LPS01) DSM 21980, in a ratio by weight of 1:1.

The cold purified water (at 5° C.) is weighed into a glass container (for example a beaker) with magnetic stirring, a thermometer, and an external ice bath and the potassium sorbate and sodium benzoate are added under stirring. The mixture is maintained under stirring at 5° C. until complete dissolution of the salts. Then the following are added, again under stirring: the two poloxamers (crystalline), in succession or in a mixture, the methionine (in powder, or in the form of micro-/nanoparticles) and the propylene glycol and cold stirring is continued for at least 15 minutes until a homogeneous appearance is obtained. The container is then placed in a refrigerator at 5° C. for at least 24 h, until obtaining complete dissolution of the ingredients.

After this, the resulting product (liquid) is packaged in 18-20 g tubes and sealed pending use/experimentation. On reaching room temperature and in contact with the skin the product transforms completely into a gel, which is rapidly absorbed following cutaneous and/or transmucosal administration. The pH of the liquid thermogel is 7.40. The gelling temperature is 21°-22° C.

The composition in the form of a thermogel of the present invention, topically administered, in particular on wrists and/or forearms, has shown an excellent cutaneous permeability, triggering a fast onset of the pharmacological action. Furthermore, thanks to the fact of going directly into circulation without passing through the gastrointestinal tract (no metabolism by the liver), it has also shown high blood levels of the active ingredient, for example, melatonin, for a prolonged period of time (slow release). This fact has made it possible to apply low dosages of the drug, thus obtaining the desired advantageous results (modest dosage/high, long-lasting activity) compared to the traditional forms of administration thereof.

For example, a study was conducted on 10 healthy volunteers with different formulations of melatonin, observing the accumulation of melatonin in the saliva over time (it is well known that melatonin spreads passively into saliva via the bloodstream, and its concentration therein represents 24%-33% of its plasma levels, that is to say, the amount of free melatonin not bound to globulins).

The following treatments were administered to the volunteers, randomly divided into 3 groups:
1. treatment with a formulation of melatonin in a reversible thermogel in accordance with the present invention (dosage 1 mg/g, single dose)
2. treatment with a formulation of melatonin in a reversible thermogel in accordance with the present invention (dosage 2 mg/g, single dose)
3. treatment with a formulation of melatonin in cream (dosage 1 mg/g, single dose)
4. treatment with melatonin in tablets (dosage 1 mg/tablet).

A saliva sample was taken at time 0 (basal) and at subsequent times (up to 7 h) after administration, using a Cortisol-Salivette® device (SARSTEDT S.r.l., Verona, IT); the melatonin levels in the saliva were determined by LC-MS (liquid chromatography-mass spectrometry) using a triple quadrupole mass spectrometer (ABSciex QTrap 3200) and substantially following the method of Khan et al. 2013.

The study showed that the formulations of melatonin in a reversible thermogel in accordance with the present invention possess an excellent thermal permeability, a fast onset of the pharmacological action and a lasting duration thereof compared to the other formulations tested.

The pharmaceutical compositions of the present invention also showed to act effectively on all of the patients treated, whereas the commonly used formulations, such as, for example, oral ones, do not act on all individuals.

Furthermore, the pharmaceutical compositions of the present invention have also shown to possess good stability over time.

The pharmaceutical compositions of the present invention are particularly recommended both for transdermal administration and transmucosal administration.

The pharmaceutical compositions of the present invention have shown to be advantageously active, or in any case very promising in the treatment of a number of disorders/diseases due to dysfunctions of physiological functions connected with circadian rhythms such as, for example, the sleep-wake balance; in the regulation of blood pressure; in eliminating of free radicals; in interacting with the immune system, for example, in the treatment of inflammatory states, in the treatment of acquired immunodeficiencies and in the treatment of viral and bacterial infectious diseases and cancer.

Furthermore, the compositions of the present invention have shown to be useful: in the treatment of sleep disorders in all the treated subjects, by favouring the quality of sleep, in particular of REM sleep; in the treatment of the autism and Down syndrome, particularly in children, by favouring their balance; in the treatment of Alzheimer's disease; in the treatment of Parkinson's disease, by improving sleep and tremors: in the treatment of the vision disorders, for example in maculopathy of the retina, via external applications in the vicinity of the eye; in the treatment of the pulmonary manifestations of cystic fibrosis in children, where they provide an anti-inflammatory action; in the treatment of prostate cancer, where they are capable of preventing the propagation thereof; in the treatment of the tumours, as anti-inflammatory, anti-tumour adjuvants.

| | | Commercial | Depositary | Deposit | Deposit | |
|---|---|---|---|---|---|---|
| No. | Name | code | institution | number | date | Depositor |
| 1 | Lactobacillus casei | LF1i | CNCM I.P. | I-785 | 21 Jul. 1988 | Anidral Srl |
| 2 | Lactobacillus gasseri | LF2i | CNCM I.P. | I-786 | 21 Jul. 1988 | Anidral Srl |
| 3 | Lactobacillus crispatus | LF3i | CNCM I.P. | I-787 | 21 Jul. 1988 | Anidral Srl |
| 4 | Lactobacillus fermentum | LF4i | CNCM I.P. | I-788 | 21 Jul. 1988 | Anidral Srl |
| 5 | Lactobacillus fermentum | LF5 | CNCM I.P. | I-789 | 21 Jul. 1988 | Anidral Srl |
| 6 | Lactobacillus casei ssp. pseudoplantarum | LFH i | CNCM I.P. | I-790 | 21 Jul. 1988 | Anidral Srl |
| 7 | Streptococcus thermophilus B39 | | BCCM LMG | LMG P-18383 | 5 May 1988 | Anidral Srl |
| 8 | Streptococcus thermophilus T003 | | BCCM LMG | LMG P-18384 | 5 May 1988 | Anidral Srl |
| 9 | Lactobacillus pentosus 9/1 ei | | BCCM LMG | LMG P-21019 | 16 Oct. 2001 | Mofin Srl |
| 0 | Lactobacillus plantarum 776/1 bi | LP 02 | BCCM LMG | LMG P-21020 | 16 Oct. 2001 | Mofin Srl |
| 11 | Lactobacillus plantarum 476LL 20 bi | LP 01 | BCCM LMG | LMG P-21021 | 16 Oct. 2001 | Mofin Srl |
| 12 | Lactobacillus plantarum PR ci | | BCCM LMG | LMG P-21022 | 16 Oct. 2001 | Mofin Srl |
| 13 | Lactobacillus plantarum 776/2 hi | | BCCM LMG | LMG P-21023 | 16 Oct. 2001 | Mofin Srl |
| 14 | Lactobacillus casei ssp. paracasei 181A/3 aiai | LPC00 | BCCM LMG | LMG P-21380 | 31 Jan. 2002 | Anidral Srl |
| 15 | Lactobacillus belonging to the acidophilus | LA 02 | BCCM LMG | LMG P-21381 | 31 Jan. 2002 | Anidral Srl |
| | group 192A/1 aai | | | | | |
| 16 | Bifidobacterium longum 175A/1 aiai | | BCCM LMG | LMG P-21382 | 31 Jan. 2002 | Anidral Srl |
| 17 | Bifidobacterium breve 195A/1 aici | | BCCM LMG | LMG P-21383 | 31 Jan. 2002 | Anidral Srl |
| 18 | Bifidobacterium lactis 32A/3 aiai | BS 01 | BCCM LMG | LMG P-21384 | 31 Jan. 2002 | Anidral Srl |
| 19 | Lactobacillus plantarum 501/2 gi | COAKTIV | BCCM LMG | LMG P-21385 | 31 Jan. 2002 | Mofin Srl |
| 20 | Lactococcus lactis ssp. lactis 504/4 ci | | BCCM LMG | LMG P-21388 | 31 Jan. 2002 | Mofin Srl |
| 21 | Lactococcus lactis ssp. lactis 501/4 hi | | BCCM LMG | LMG P-21387 | 15 Mar. 2002 | Mofin Srl |
| 22 | Lactococcus lactis ssp. lactis 501/4 ci | | BCCM LMG | LMG P-21388 | 31 Jan. 2002 | Mofin Srl |
| 23 | Lactobacillus plantarum 501/4 li | | BCCM LMG | LMG P-21389 | 15 Mar. 2002 | Mofin Srl |
| 24 | Lactobacillus acidophilus | LA08 | BCCM LMG | LMG P-26144 | 3 Nov. 2010 | Probiotical |
| | | | | | | SpA |
| 25 | Lactobacillus paracasei ssp. paracasei | LPC10 | BCCM LMG | LMG P-26143 | 3 Nov. 2010 | Probiotical |
| | | | | | | SpA |
| 26 | Streptococcus thennophilus | GB1 | DSMZ | DSM 16506 | 18 Jun. 2004 | Anidral Srl |
| 27 | Streptococcus thermophilus | GB5 | DSMZ | DSM 16507 | 18 Jun. 2004 | Anidral Srl |
| 28 | Streptococcus thermophilus | Y02 | DSMZ | DSM 16590 | 20 Jul. 2004 | Anidral Srl |
| 29 | Streptococcus thermophilus | Y03 | DSMZ | DSM 16591 | 20 Jul. 2004 | Anidral Srl |
| 30 | Streptococcus thermophilus | Y04 | DSMZ | DSM 16592 | 20 Jul. 2004 | Anidral Srl |
| 31 | Streptococcus thermophilus | YO5 | DSMZ | DSM 16593 | 20 Jul. 2004 | Anidral Srl |
| 32 = 56 | Bifidobacterium adolescentis | BA 03 | DSMZ | DSM 16594 | 21 Jul. 2004 | Anidral Srl |
| 33 | Bifidobacterium adolescentis | BA 04 | DSMZ | DSM 16595 | 21 Jul. 2004 | Anidral Srl |
| 34 | Bifidobacterium breve | BR 04 | DSMZ | DSM 16596 | 21 Jul. 2004 | Anidral Srl |
| 35 | Bifidobacterium pseudocatenulatum | BP 01 | DSMZ | DSM 16597 | 21 Jul. 2004 | Anidral Srl |
| 36 | Bifidobacterium pseudocatenulatum | BP 02 | DSMZ | DSM 16598 | 21 Jul. 2004 | Anidral Srl |
| 37 | Bifidobacterium longum | BL 03 | DSMZ | DSM 16603 | 20 Jul. 2004 | Anidral Srl |
| 38 | Bifidobacterium breve | BR 03 | DSMZ | DSM 16604 | 20 Jul. 2004 | Anidral Srl |
| 39 | Lactobacillus casei ssp. rhamnosus | LR 04 | DSMZ | DSM 16605 | 20 Jul. 2004 | Anidral Srl |
| 40 | Lactobacillus delbrueckii ssp. bulgaricus | LDB 01 | DSMZ | DSM 16606 | 20 Jul. 2004 | Anidral Srl |
| 41 | Lactobacillus delbrueckii ssp. bulgaricus | LDB 02 | DSMZ | DSM 16607 | 20 Jul. 2004 | Anidral Srl |
| 42 | Staphylococcus xylosus | SX 01 | DSMZ | DSM 17102 | 1 Feb. 2005 | Anidral Srl |
| 43 = 57 | Bifidobacterium adolescentis | BA 02 | DSMZ | DSM 17103 | 1 Feb. 2005 | Anidral Srl |
| 44 | Lactobacillus plantarum | LP 07 | DSMZ | DSM 17104 | 1 Feb. 2005 | Anidral Srl |
| 45 | Streptococcus thermophilus | YO8 | DSMZ | DSM 17843 | 21 Dec. 2005 | Anidral Srl |
| 46 | Streptococcus thermophilus | YO9 | DSMZ | DSM 17844 | 21 Dec. 2005 | Anidral Srl |
| 47 | Streptococcus thermophilus | YO100 | DSMZ | DSM 17845 | 21 Dec. 2005 | Anidral Srl |
| 48 | Lactobacillus fermentum | LF06 | DSMZ | DSM 18295 | 24 May 2006 | Anidral Srl |
| 49 | Lactobacillus fermentum | LF07 | DSMZ | DSM 18296 | 24 May 2006 | Anidral Srl |
| 50 | Lactobacillus fermentum | LF08 | DSMZ | DSM 18297 | 24 May 2006 | Anidral Srl |
| 51 | Lactobacillus fermentum | LF09 | DSMZ | DSM 18298 | 24 May 2006 | Anidral Srl |
| 52 | Lactobacillus gasseri | LGS01 | DSMZ | DSM 18299 | 24 May 2006 | Anidral Srl |
| 53 | Lactobacillus gasseri | LGA02 | DSMZ | DSM 18300 | 24 May 2006 | Anidral Srl |
| 54 | Lactobacillus gasseri | LGS03 | DSMZ | DSM 18301 | 24 May 2006 | Anidral Srl |
| 55 | Lactobacillus gasseri | LGS04 | DSMZ | DSM 18302 | 24 May 2006 | Anidral Srl |
| 56 = 32 | Bifidobacterium adolescentis EI-3 | BA 03 | DSMZ | DSM 18350 | 15 Jun. 2006 | Anidral Srl |
| | Bifidobacterium catenulatum | | | | | |
| | sp./pseudocatenulatum EI-3I, ID 09-255 | | | | | |
| 57 = 43 | Bifidobacterium adolescentis EI-15 | BA 02 | DSMZ | DSM 18351 | 15 Jun. 2006 | Anidral Srl |
| 58 | Bifidobacterium adolescentis EI-18 | BA 05 | DSMZ | DSM 18352 | 15 Jun. 2006 | Anidral Srl |
| | Bifidobacterium animalis subsp. lacts EI-18, | | | | | |
| | ID 09-256 | | | | | |
| 59 | Bifidobacterium catenulatum EI-20 | BC 01 | DSMZ | DSM 18353 | 15 Jun. 2006 | Anidral Srl |
| 60 | Streptococcus thermophilus FRai | MO1 | DSMZ | DSM 18613 | 13 Sep. 2006 | Mofin Srl |
| 61 | Streptococcus thermophilus LB2bi | MO2 | DSMZ | DSM 18614 | 13 Sep. 2006 | Mofin Srl |
| 62 | Streptococcus thermophilus LRci | MO3 | DSMZ | DSM 18615 | 13 Sep. 2006 | Mofin Srl |
| 63 | Streptococcus thermophilus FP4 | MO4 | DSMZ | DSM 18616 | 13 Sep. 2006 | Mofin Srl |
| 64 | Streptococcus thermophilus ZZ5F8 | MO5 | DSMZ | DSM 18617 | 13 Sep. 2006 | Mofin Srl |
| 65 | Streptococcus thermophilus TEO4 | MO6 | DSMZ | DSM 18618 | 13 Sep. 2006 | Mofin Srl |
| 66 | Streptococcus thermophilus S1ci | MO7 | DSMZ | DSM 18619 | 13 Sep. 2006 | Mofin Srl |
| 67 | Streptococcus thermophilus 641bi | MO8 | DSMZ | DSM 18620 | 13 Sep. 2006 | Mofin Srl |
| 68 | Streptococcus thermophilus 277A/1ai | MO9 | DSMZ | DSM 18621 | 13 Sep. 2006 | Mofin Srl |
| 69 | Streptococcus thermophilus 277A/2ai | MO10 | DSMZ | DSM 18622 | 13 Sep. 2006 | Mofin Srl |
| 70 | Streptococcus thermophilus IDC11 | MO11 | DSMZ | DSM 18623 | 13 Sep. 2006 | Mofin Srl |
| 71 | Streptococcus thermophilus ML3di | MO14 | DSMZ | DSM 18624 | 13 Sep. 2006 | Mofin Srl |
| 72 | Streptococcus thermophilus TEO3 | MO15 | DSMZ | DSM 18625 | 13 Sep. 2006 | Mofin Srl |
| 73 | Streptococcus thermophilus G62 | GG1 | DSMZ | DSM 19057 | 21 Feb. 2007 | Mofin Srl |
| 74 | Streptococcus thermophilus G1192 | GG2 | DSMZ | DSM 19058 | 21 Feb. 2007 | Mofin Srl |
| 75 | Streptococcus thermophilus GB18 | GG3 | DSMZ | DSM 19059 | 21 Feb. 2007 | Mofin Srl |
| | | MO2 | | | | |
| 76 | Streptococcus thermophilus CCR21 | GG4 | DSMZ | DSM 19660 | 21 Feb. 2007 | Mofin Srl |
| 77 | Streptococcus thermophilus G92 | GG5 | DSMZ | DSM 19061 | 21 Feb. 2007 | Mofin Srl |
| 78 | Streptococcus thermophilus G69 | GG6 | DSMZ | DSM 19062 | 21 Feb. 2007 | Mofin Srl |
| 79 | Streptococcus thermophilus | YO 10 | DSMZ | DSM 19063 | 21 Feb. 2007 | Anidral Srl |
| 80 | Streptococcus thermophilus | YO 11 | DSMZ | DSM 19064 | 21 Feb. 2007 | Anidral Srl |
| 81 | Streptococcus thermophilus | YO 12 | DSMZ | DSM 19065 | 21 Feb. 2007 | Anidral Srl |
| 82 | Streptococcus thermophilus | YO 13 | DSMZ | DSM 19066 | 21 Feb. 2007 | Anidral Srl |
| 83 | Weissella ssp. WSP 01 | EX | DSMZ | DSM 19067 | 21 Feb. 2007 | Anidral Srl |
| 84 | Weissella ssp. WSP 02 | EX | DSMZ | DSM 19068 | 21 Feb. 2007 | Anidral Srl |
| 85 | Lactobacillus ssp. WSP 03 | EX | DSMZ | DSM 19069 | 21 Feb. 2007 | Anidral Srl |
| 86 | Lactobacillus plantarum LP 09 | OY | DSMZ | DSM 19070 | 21 Feb. 2007 | Anidral Srl |
| 87 | Lactobacillus plantarum LP 10 | OY | DSMZ | DSM 19071 | 21 Feb. 2007 | Anidral Srl |
| 88 | Lactococcus lactis | NS 01 | DSMZ | DSM 19072 | 21 Feb. 2007 | Anidral Srl |
| 89 | Lactobacillus fermentum | LF 10 | DSMZ | DSM 19187 | 20 Mar. 2007 | Anidral Srl |
| 90 | Lactobacillus fermentum | LF 11 | DSMZ | DSM 19188 | 20 Mar. 2007 | Anidral Srl |
| 91 | Lactobacillus casei ssp. rhamnosus | LR05 | DSMZ | DSM 19739 | 27 Sep. 2007 | Anidral Srl |
| 92 | Bifidobacterium bifidum | BB01 | DSMZ | DSM 19818 | 30 Oct. 2007 | Anidral Srl |
| 93 | Lactobacillus delbrueckii subsp. | Lb | DSMZ | DSM 19948 | 28 Nov. 2007 | Anidral Srl |
| | bulgaricus LD 01 | | | | | |
| 94 | Lactobacillus delbrueckii subsp. | Lb | DSMZ | DSM 19949 | 28 Nov. 2007 | Anidral Srl |
| | bulgaricus LD 02 | | | | | |
| 95 | Lactobacillus delbrueckii subsp. | Lb | DSMZ | DSM 19950 | 28 Nov. 2007 | Anidral Srl |
| | bulgaricus LD 03 | | | | | |
| 96 | Lactobacillus delbrueckii subsp. | Lb | DSMZ | DSM 19951 | 28 Nov. 2007 | Anidral Srl |
| | bulgalicus LD 04 | | | | | |
| 97 | Lactobacillus delbrueckii subsp. | Lb | DSMZ | DSM 19952 | 28 Nov. 2007 | Anidral Srl |
| | bulgaricus LD 05 | | | | | |
| 98 | Bifidobacterium pseudocatenulatum | B660 | DSMZ | DSM 21444 | 13 May 2008 | Probiotical |
| | | | | | | SpA |
| 99 | Lactobacillus acidophilus | LA02 | DSMZ | DSM 21717 | 6 Aug. 2008 | Probiotical |
| | | | | | | SpA |
| 100 | Lactobacillus paracasei | LPC 08 | DSMZ | DSM 21718 | 6 Aug. 2008 | Probiotical |
| | | | | | | SpA |
| 101 | Lactobacillus pentosus | LPS 01 | DSMZ | DSM 21980 | 14 Nov. 2008 | Probiotical |
| | | | | | | SpA |
| 102 | Lactobacillus rahmnosus | LR 06 | DSMZ | DSM 21981 | 14 Nov. 2008 | Probiotical |
| | | | | | | SpA |
| 103 | Lactobacillus delbrueckii ssp. delbrueckii | DSMZ 20074 | DSMZ | DSM 22106 | 10 Dec. 2008 | Probiotical |
| | | | | | | SpA |
| 104 | Lactobacillus plantarum | LP1 | DSMZ | DSM 22107 | 10 Dec. 2008 | Probiotical |
| | | | | | | SpA |
| 105 | Lactobacillus salivarius | LS01 | DSMZ | DSM 22775 | 23 Jul. 2009 | Probiotical |
| | | | | | | SpA |
| 106 | Lactobacillus salivarius | LS03 | DSMZ | DSM 22776 | 23 Jul. 2009 | Probiotical |
| | | | | | | SpA |
| 107 | Bifidobacterium bifidum | BB01 | DSMZ | DSM 22892 | 28 Aug. 2009 | Probiotical |
| | | | | | | SpA |
| 108 | Bifidobacterium bifidum | | DSMZ | DSM 22893 | 28 Aug. 2009 | Probiotical |
| | | | | | | SpA |
| 109 | Bifidobacterium bifidum | BB03 | DSMZ | DSM 22894 | 28 Aug. 2009 | Probiotical |
| | | | | | | SpA |
| 110 | Bifidobacterium lactis | BS05 | DSMZ | DSM 23032 | 13 Oct. 2009 | Probiotical |
| | | | | | | SpA |
| 111 | Lactobacillus acidophilus | LA 06 | DSMZ | DSM 23033 | 13 Oct. 2009 | Probiotical |
| | | | | | | SpA |
| 112 | Lactobacillus brevis | LBR01 | DSMZ | DSM 23034 | 13 Oct. 2009 | Probiotical |
| | | | | | | SpA |
| 113 | Bifidobacterium animalis ssp. lactis | BS06 | DSMZ | DSM 23224 | 12 Jan. 2010 | Probiotical |
| | | | | | | SpA |
| 114 | Bifidobacterium longum | L04 | DSMZ | DSM 23233 | 12 Jan. 2010 | Probiotical |
| | | | | | | SpA |
| 115 | Bifidobacterium longum | BL05 | DSMZ | DSM 23234 | 12 Jan. 2010 | Probiotical |
| | | | | | | SpA |
| 116 | Bifidobacterium bifidum | MB 109 | DSMZ | DSM 23731 | 29 Jun. 2010 | Probiotical |
| | | | | | | SpA |
| 117 | Bifidobacterium breve | MB 113 | DSMZ | DSM 23732 | 29 Jun. 2010 | Probiotical |
| | | | | | | SpA |
| 118 | Bifidobacterium lactis | MB 2409 | DSMZ | DSM 23733 | 29 Jun. 2010 | Probiotical |
| | | | | | | SpA |
| 119 | Lactobacillus reuteri | LRE01 | DSMZ | DSM 23877 | 5 Aug. 2010 | Probiotical |
| | | | | | | SpA |
| 120 | Lactobacillus reuteri | LRE02 | DSMZ | DSM 23878 | 5 Aug. 2010 | Probiotical |
| | | | | | | SpA |
| 121 | Lactobacillus reuteri | LRE03 | DSMZ | DSM 23879 | 5 Aug. 2010 | Probiotical |
| | | | | | | SpA |
| 122 | Lactobacillus reuteri | LRE04 | DSMZ | DSM 23880 | 5 Aug. 2010 | Probiotical |
| | | | | | | SpA |
| 123 | Lactobacillus paracasei ssp. paracasei | LPC09 | DSMZ | DSM 24243 | 23 Nov. 2010 | Probiotical |
| | | | | | | SpA |
| 124 | Lactobacillus acidophilus | LA 07 | DSMZ | DSM 24303 | 23 Nov. 2010 | Probiotical |
| | | | | | | SpA |
| 125 | Bifidobacterium bifidum | BB04 | DSMZ | DSM 24437 | 4 Jan. 2011 | Probiotical |
| | | | | | | SpA |
| 126 | Lactobacillus crispatus | CRL 1251 | DSMZ | DSM 24438 | 4 Jan. 2011 | Probiotical |
| | | | | | | SpA |
| 127 | Lactobacillus crispatus | CRL 1266 | DSMZ | DSM 24439 | 4 Jan. 2011 | Probiotical |
| | | | | | | SpA |
| 128 | Lactobacillus paracasei | CRL 1289 | DSMZ | DSM 24440 | 4 Jan. 2011 | Probiotical |
| | | | | | | SpA |
| 129 | Lactobacillus Salivarius | CRL 1328 | DSMZ | DSM 24441 | 4 Jan. 2011 | Probiotical |
| | | | | | | SpA |
| 130 | Lactobacillus gasseri | CRL 1259 | DSMZ | DSM 24512 | 25 Jan. 2011 | Probiotical |
| | | | | | | SpA |
| 131 | Lactobacillus acidophilus | CRL 1294 | DSMZ | DSM 24513 | 25 Jan. 2011 | Probiotical |
| | | | | | | SpA |
| 132 | Lactobacillus salivarius | LS04 | DSMZ | DSM 24618 | 2 Mar. 2011 | Probiotical |
| | | | | | | SpA |
| 133 | Lactobacillus crispatus | LCR01 | DSMZ | DSM 24619 | 2 Mar. 2011 | Probiotical |
| | | | | | | SpA |
| 134 | Lactobacillus crispatus | LCR02 | DSMZ | DSM 24620 | 2 Mar. 2011 | Probiotical |
| | | | | | | SpA |
| 135 | Lactobacillus acidophilus | LA09 | DSMZ | DSM 24621 | 2 Mar. 2011 | Probiotical |
| | | | | | | SpA |
| 136 | Lactobacillus gasseri | LGS05 | DSMZ | DSM 24622 | 2 Mar. 2011 | Probiotical |
| | | | | | | SpA |
| 137 | Lactobacillus paracasei | LPC11 | DSMZ | DSM 24623 | 2 Mar. 2011 | Probiotical |
| | | | | | | SpA |
| 138 | Bifidobacterium infantis | BI 02 | DSMZ | DSM 24687 | 29 Mar. 2011 | Probiotical |
| | | | | | | SpA |
| 139 | Bifidobacterium bifidum | BB 06 | DSMZ | DSM 24688 | 29 Mar. 2011 | Probiotical |
| | | | | | | SpA |
| 140 | Bifidobacterium longum | BL 06 | DSMZ | DSM 24689 | 29 Mar. 2011 | Probiotical |
| | | | | | | SpA |
| 141 | Bifidobacterium lactis | BS 07 | DSMZ | DSM 24690 | 29 Mar. 2011 | Probiotical |
| | | | | | | SpA |
| 142 | Bifidobacterium longum | PCB133 | DSMZ | DSM 24691 | 29 Mar. 2011 | Probiotical |
| | | | | | | SpA |
| 143 | Bifidobacterium breve | B632 | DSMZ | DSM 24706 | 7 Apr. 2011 | Probiotical |
| | | | | | | SpA |
| 144 | Bifidobacteriura breve | B2274 | DSMZ | DSM 24707 | 7 Apr. 2011 | Probiotical |
| | | | | | | SpA |
| 145 | Bifidobacterium breve | B7840 | DSMZ | DSM 24708 | 7 Apr. 2011 | Probiotical |
| | | | | | | SpA |
| 146 | Bifidobacterium longum | B1975 | DSMZ | DSM 24709 | 7 Apr. 2011 | Probiotical |
| | | | | | | SpA |
| 147 | Lactobacillus salivarius | DLV1 | DSMZ | DSM 25138 | 2 Sep. 2011 | Probiotical |
| | | | | | | SpA |
| 148 | Lactobacillus | LRE05 | DSMZ | DSM 25139 | 2 Sep. 2011 | Probiotical |
| | reuteri | | | | | SpA |
| 149 | Lactobacillus | LRE06 | DSMZ | DSM 25140 | 2 Sep. 2011 | Probiotical |
| | reuteri | | | | | SpA |
| 150 | Lactobacillus | RC 14 | DSMZ | DSM 25141 | 2 Sep. 2011 | Probiotical |
| | reuteri | | | | | SpA |
| 151 | Streptococcus thermophilus | ST 10 | DSMZ | DSM 25246 | 19 Sep. 2011 | Probiotical |
| | | | | | | SpA |
| 152 | Streptococcus thermophilus | ST 11 | DSMZ | DSM 25247 | 19 Sep. 2011 | Probiotical |
| | | | | | | SpA |
| 153 | Streptococcus thermophilus | ST 12 | DSMZ | DSM 25282 | 20 Oct. 2011 | Probiotical |
| | | | | | | SpA |
| 154 | Lactobacillus salivarius | DLV8 | DSMZ | DSM 25545 | 12 Jan. 2012 | Probiotical |
| | | | | | | SpA |
| 155 | Bifidobacterium longum | DLBL 07 | DSMZ | DSM 25669 | 16 Feb. 2012 | Probiotical |
| | | | | | | SpA |
| 156 | Bifidobacterium longum | DLBL 08 | DSMZ | DSM 25670 | 16 Feb. 2012 | Probiotical |
| | | | | | | SpA |
| 157 | Bifidobacterium longum | DLBL 09 | DSMZ | DSM 25671 | 16 Feb. 2012 | Probiotical |
| | | | | | | SpA |
| 158 | Bifidobacterium longum | DLBL 10 | DSMZ | DSM 25672 | 16 Feb. 2012 | Probiotical |
| | | | | | | SpA |
| 159 | Bifidobacterium longum | DLBL 11 | DSMZ | DSM 25673 | 16 Feb. 2012 | Probiotical |
| | | | | | | SpA |
| 160 | Bifidobacterium longum | DLBL 12 | DSMZ | DSM 25674 | 16 Feb. 2012 | Probiotical |
| | | | | | | SpA |
| 161 | Bifidobacterium longum | DLBL13 | DSMZ | DSM 25675 | 16 Feb. 2012 | Probiotical |
| | | | | | | SpA |
| 162 | Bifidobacterium longum | DLBL 14 | DSMZ | DSM 25676 | 16 Feb. 2012 | Probiotical |
| | | | | | | SpA |
| 163 | Bifidobacterium longum | DLBL 15 | DSMZ | DSM 25677 | 16 Feb. 2012 | Probiotical |
| | | | | | | SpA |
| 164 | Bifidobacterium longum | DLBL 16 | DSMZ | DSM 25678 | 16 Feb. 2012 | Probiotical |
| | | | | | | SpA |
| 165 | Bifidobacterium longum | DLBL 17 | DSMZ | DSM 25679 | 16 Feb. 2012 | Probiotical |
| | | | | | | SpA |
| 166 | Lactobacillus johnsonii | DLLJO 01 | DSMZ | DSM 25680 | 16 Feb. 2012 | Probiotical |
| | | | | | | SpA |
| 167 | Lactobacillus rhamnosus | DLLR 07 | DSMZ | DSM 25681 | 16 Feb. 2012 | Probiotical |
| | | | | | | SpA |
| 168 | Lactobacillus rhamnosus | DLLR 08 | DSMZ | DSM 25632 | 16 Feb. 2012 | Probiotical |
| | | | | | | SpA |
| 169 | Lactobacillus reuteri | DLLRE 07 | DSMZ | DSM 25683 | 16 Feb. 2012 | Probiotical |
| | | | | | | SpA |
| 170 | Lactobacillus reuteri | DLLRE 08 | DSMZ | DSM 25684 | 16 Feb. 2012 | Probiotical |
| | | | | | | SpA |
| 171 | Lactobacillus reuteri | DLLRE 09 | DSMZ | DSM 25685 | 16 Feb. 2012 | Probiotical |
| | | | | | | SpA |
| 172 | Bifidobacterium longum | DLBL 18 | DSMZ | DSM 25708 | 24 Feb. 2012 | Probiotical |
| | | | | | | SpA |
| 173 | Bifidobacterium infantis | BI 03 | DSMZ | DSM 25709 | 24 Feb. 2012 | Probiotical |
| | | | | | | SpA |
| 174 | Lactobacillus plantarum | LP 09 | DSMZ | DSM 25710 | 24 Feb. 2012 | Probiotical |
| | | | | | | SpA |
| 175 | Bifidobacterium longum | DLBL 19 | DSMZ | DSM 25717 | 1 Mar. 2012 | Probiotical |
| | | | | | | SpA |
| 176 | Bifidobacterium longum | DLBL 20 | DSMZ | DSM 25718 | 1 Mar. 2012 | Probiotical |
| | | | | | | SpA |
| 177 | Lactobacillus salivarius | LS 05 | DSMZ | DSM 26036 | 6 Jun. 2012 | Probiotical |
| | | | | | | SpA |
| 178 | Lactobacillus salivatius | LS 06 | DSMZ | DSM 26037 | 6 Jun. 2012 | Probiotical |
| | | | | | | SpA |
| 179 | Lactobacillus pentosus | LPS 02 | DSMZ | DSM 26038 | 6 Jun. 2012 | Probiotical |
| | | | | | | SpA |
| 180 | Bifidobacterium pseudolongum | BPS 01 | DSMZ | DSM 26456 | 2 Oct. 2012 | Probiotical |
| | ssp. globosum | | | | | SpA |
| 181 | Lactobacillus fermentum | LF15 | DSMZ | DSM 26955 | 1 Mar. 2013 | Probiotical |
| | | | | | | SpA |
| 182 | Lactobacillus fermentum | LF16 | DSMZ | DSM 26956 | 1 Mar. 2013 | Probiotical |
| | | | | | | SpA |
| 183 | Lactobacillus casei | LC03 | DSMZ | DSM 27537 | 24 Jul. 2013 | Probiotical |
| | | | | | | SpA |
| 184 | Lactobacillus crispatus | LCR03 | DSMZ | DSM 27538 | 24 Jul. 2013 | Probiotical |
| | | | | | | SpA |
| 185 | Lactobacillus jensenii | LJE01 | DSMZ | DSM 27539 | 24 Jul. 2013 | Probiotical |
| | | | | | | SpA |
| 186 | Lactobacillus helveticus ID 922 | LH01 | DSMZ | DSM 28153 | 4 Dec. 2013 | Probiotical |
| | | | | | | SpA |
| 187 | Lactobacillus helveticus ID 923 | LH02 | DSMZ | DSM 28154 | 4 Dec. 2013 | Probiotical |
| | | | | | | SpA |
| 188 | Lactococcus lactis ssp. cremoris ID 1612 | LLC02 | DSMZ | DSM 28155 | 4 Dec. 2013 | Probiotical |
| | | | | | | SpA |
| 189 | Lactococcus lactis ssp. cremoris ID 1252 | LLC03 | DSMZ | DSM 28156 | 4 Dec. 2013 | Probiotical |
| | | | | | | SpA |
| 190 | Lactococcus lactis ssp. Lactis ID 1254 | LLL01 | DSMZ | DSM 28157 | 4 Dec. 2013 | Probiotical |
| | | | | | | SpA |
| 191 | Bifidobacterium longum | BL 01 | DSMZ | DSM 28173 | 11 Dec. 2013 | Probiotical |
| | | | | | | SpA |
| 192 | Bifidobacterium lungum | BL 02 | DSMZ | DSM 28174 | 11 Dec. 2013 | Probiotical |
| | | | | | | SpA |
| 193 | Bifidobaterium animalis ssp. lactis | Bb1 | DSMZ | DSM 17850 | 23 Dec. 2005 | BiaMan Srl |
| 194 | Streptococcus thermophilus | ST 16 BM | DSMZ | DSM 19526 | 13 Jul. 2007 | BioMan Srl |
| 195 | Bifidobacterium infantis | BI 04 | DSMZ | DSM 28651 | 8 Apr. 2014 | Probiotical |
| | | | | | | SpA |
| 196 | Bifidobacterium infantis | BI 05 | DSMZ | DSM 28652 | 8 Apr. 2014 | Probiotical |
| | | | | | | SpA |
| 197 | Streptococcus thermophilus | ST 15 | DSMZ | DSM 28911 | 11 Jun. 2014 | Probiotical |
| | | | | | | SpA |
| 198 | Streptococcus thermophilus | ST 16 | DSMZ | DSM 28912 | 11 Jun. 2014 | Probiotical |
| | | | | | | SpA |
| 199 | Streptococcus thermophilus | ST 17 | DSMZ | DSM 28913 | 11 Jun. 2014 | Probiotical |
| | | | | | | SpA |
| 200 | Lactobacillus fermentum | LF18 | DSMZ | DSM 29197 | 30 Jul. 2014 | Probiotical |
| | | | | | | SpA |
| 201 | Lactobacillus fermentum | LF19 | DSMZ | DSM 29198 | 30 Jul. 2014 | Probiotical |
| | | | | | | SpA |
| 202 | Leuconostoc sp. | LM01 | DSMZ | DSM 29372 | 10 Sep. 2014 | Mofin Srl |
| 203 | Leuccnostoc sp. | LM10 | DSMZ | DSM 29373 | 10 Sep. 2014 | Mofin Srl |
| 204 | Leuconostoc sp. | LM11 | DSMZ | DSM 29374 | 10 Sep. 2014 | Mofin Srl |
| 205 | Leuconostoc sp. | LM12 | DSMZ | DSM 29375 | 10 Sep. 2014 | Mofin Srl |
| 206 | Lactobacillusplantarum | LP10 | DSMZ | DSM 29389 | 10 Sep. 2014 | Mofin Srl |
| 207 | Lactobacillusplantarum | LP11 | DSMZ | DSM 29390 | 10 Sep. 2014 | Mofin Srl |
| 208 | Lactobacillusplantarum | LP12 | DSMZ | DSM 29400 | 10 Sep. 2014 | Mofin Srl |
| 209 | Lactobacillusplantarum | LP13 | DSMZ | DSM 29401 | 10 Sep. 2014 | Mofin Srl |
| 210 | Lactobacillus pentosus | LPS03 | DSMZ | DSM 29402 | 10 Sep. 2014 | Mofin Srl |
| 211 | Lactobacillus reuteri | LRE10 | DSMZ | DSM 29403 | 10 Sep. 2014 | Mofin Srl |
| 212 | Lactobacillus brevis | LBR02 | DSMZ | DSM 29404 | 10 Sep. 2014 | Mofin Srl |
| 213 | Lactobacillus salivarius | LS 07 | DSMZ | DSM 29476 | 9 Oct. 2014 | Probiotical |
| | | | | | | SpA |
| 214 | Bifidobacterium breve | BR 05 | DSMZ | DSM 29494 | 9 Oct. 2014 | Probiotical |
| | | | | | | SpA |
| 215 | Lactococcus lactis ssp. cremoris | LCC02 | DSMZ | DSM 29536 | 22 Oct. 2014 | Probiotical |
| | | | | | | SpA |
| 216 | Bifidobacterium longum | BL 21 | DSMZ | DSM 29884 | 15 Jan. 2015 | Probiotical |
| | | | | | | SpA |
| 217 | Lactobacillus rhamnosus | LR 09 | DSMZ | DSM 29885 | 15 Jan. 2015 | Probiotical |
| | | | | | | SpA |
| 218 | Lactobacillus kefiri | LKE01 | DSMZ | DSM 32027 | 8 Apr. 2015 | Probiotical |
| | | | | | | SpA |
| 219 | Lactobacillus kefiri | LKE02 | DSMZ | DSM 32056 | 29 May 2015 | Probiotical |
| | | | | | | SpA |
| 220 | Lactobacillus acidophilus | LA10 | DSMZ | DSM 32075 | 3 Jul. 2015 | Probiotical |
| | | | | | | SpA |
| 221 | Lactobacillus kefiranofaciens | LKR01 | DSMZ | DSM 32076 | 3 Jul. 2015 | Probiotical |
| | | | | | | SpA |
| 222 | Lactobacillus kefiri | LKF01 | DSMZ | DSM 32079 | 10 Jul. 2015 | Probiotical |
| | | | | | | SpA |
| 223 | Lactobaciullus kefiri | LKF02 | DSMZ | DSM 32080 | 10 Jul. 2015 | Probiotical |
| | | | | | | SpA |
| 224 | Streptococcus thermophilus | ST18 | DSMZ | DSM 32134 | 3 Sep. 2015 | Mofin S.r.l. |
| 225 | Streptococcus thermophilus | ST19 | DSMZ | DSM 32135 | 3 Sep. 2015 | Mofin S.r.l. |
| 226 | Streptococcus thermophilus | ST20 | DSMZ | DSM 32136 | 3 Sep. 2015 | Mofin S.r.l. |
| 227 | Streptococcus thermophilus | ST21 | DSMZ | DSM 32137 | 3 Sep. 2015 | Mofin S.r.l. |
| 228 | Streptococcus thermophilus | ST22 | DSMZ | DSM 32138 | 3 Sep. 2015 | Mofin S.r.l. |
| 229 | Streptococcus thermophilus | ST23 | DSMZ | DSM 32139 | 3 Sep. 2015 | Mofin S.r.l. |
| 230 | Streptococcus thermophilus | ST24 | DSMZ | DSM 32140 | 3 Sep. 2015 | Mofin S.r.l. |
| 231 | Lactobacillus salivarius | LS02 | DSMZ | DSM 32204 | 13 Nov. 2015 | Probiotical |
| | | | | | | SpA |
| 232 | Weissella confusa | WC01 | DSMZ | DSM 32156 | 22 Sep. 2015 | Mofin S.r.l. |
| 233 | Weissella confusa | WC02 | DSMZ | DSM 32157 | 22 Sep. 2015 | Mofin S.r.l. |
| 234 | Lactobacillus curvatus | LCU01 | DSMZ | DSM 32160 | 22 Sep. 2015 | Mofin S.r.l. |
| 235 | Lactobacillus plantarum | LMC1 | DSMZ | DSM 32252 | 29 Sep. 2016 | Probiotical |
| | | | | | | SpA |
| 236 | Lactobacillus reuteri | LMC3 | DSMZ | DSM 32253 | 29 Sep. 2016 | Probiotical |
| | | | | | | SpA |
| 237 | Lactobacillus parasei | LMC4 | DSMZ | DSM 32254 | 29 Sep. 2016 | Probiotical |
| | | | | | | SpA |
| 238 | Lactobacillus reuteri | LMC5 | DSMZ | DSM 32255 | 29 Sep. 2016 | Probiotical |
| | | | | | | SpA |
| 239 | Lactobacillus rhamnosus | LMC6 | DSMZ | DSM 32256 | 29 Sep. 2016 | Probiotical |
| | | | | | | SpA |
| 240 | Lactobacillus rhamnosus | LMC7 | DSMZ | DSM 32257 | 29 Sep. 2016 | Probiotical |
| | | | | | | SpA |
| 241 | Lactobacillus paracasei | LMC8 | DSMZ | DSM 32258 | 29 Sep. 2016 | Probiotical |
| | | | | | | SpA |
| 242 | Lactobacillus reuteri | LMC9 | DSMZ | DSM 32259 | 29 Sep. 2016 | Probiotical |
| | | | | | | SpA |
| 243 | Lactobacillus rhamnosus | LMC10 | DSMZ | DSM 32260 | 29 Sep. 2016 | Probiotical |
| | | | | | | SpA |
| 244 | Lactobacillus fermentum | LF25 | DSMZ | DSM 32275 | 15 Mar. 2016 | Probiotical |
| | | | | | | SpA |
| 245 | Lactobacillus fermentum | LF5 | DSMZ | DSM 32277 | 18 Mar. 2016 | Probiotical |
| | | | | | | SpA |
| 246 | Lactobacillus fermentum | LF20 | DSMZ | DSM 32288 | 14 Apr. 2016 | Probiotical |
| | | | | | | SpA |

### Materials and Methods

### Experimental Protocol

The experiment was conducted on a group of 6 volunteers, all female and aged between 29 and 41 years (mean age 34.5 years).

Each volunteer tested 5 formulations of melatonin (gel 0.1%, gel 0.2%, gel 1%, cream, Circadin 2 mg), with a wash-out period of one week after each application.

The saliva samples were taken according to the following experimental scheme:

### h 10.00 Basal

h 10.30 30′
h 11.00 1 h
h 13.00 3 h
h 16.00 6 h

The saliva was collected with Salivette (Sarstedt), following the instructions attached thereto (mouth rinsing before each sampling, chewing on the swab for 1 minute). The saliva was extracted from the swab by 2 min centrifugation at 4000 rpm, then divided into 300 μl aliquots and frozen at −20° C. until the time of analysis.

### Data Analysis

The melatonin assays were performed with the ELISA (Enzyme-Linked Immunosorbent Assay) technique, using the Melatonin (direct) Saliva kit (SLV-4779-DRG Instruments, Germany). The analytical sensitivity and inter- and intra-assay coefficients of variation are the following: 0.3 pg/ml, 7.6-13.0% and 6.1-10.8%.

The kit in question is certified for in-vitro diagnostic use.

| Patient | | | | | | |
|---|---|---|---|---|---|---|
| ID | Gel 0.1% 1 mg/g | | | | | |
| 1 | 0.42 | 6.65 | 7.91 | 18.2 | 14.6 | 37.76 |
| 2 | 0.33 | 4.88 | 4.22 | 4.72 | 2.33 | 16.48 |
| 3 | 0.1 | 1.6 | 1.62 | 7.55 | 2.09 | 10.94 |
| 4 | 1.93 | 17.9 | 22.4 | 18.7 | 9.46 | 70.39 |
| 5 | 0.1 | 5.67 | 9.59 | 2.43 | 1.67 | 19.46 |
| 6 | 0.14 | 14.4 | 8.35 | 10.10 | 8.70 | 71.56 |
| | Basal | 30′ | 1 h | 3 h | 6 h | total |
| | | | | | Total 47.87 | 226.04 |
| | | | | | | mean = 37.67 |

1. A composition in the form of a gel for topical use comprising:
an effective amount of an active substance selected from the group comprising or, alternatively, consisting of:
(i) at least one microorganism selected from the group comprising or, alternatively, consisting of: live lactic bacteria; live bifidobacteria, biologically active bacteria or active bacterial components, extracts or cell components, bacterial enzymes, tyndallized bacteria, lysed bacteria, sonicated bacteria and peptidoglycans; and
(ii) melatonin and/or the natural and/or synthetic derivatives thereof; and
a vehicle or carrier comprising water, a thickening viscous matrix comprising or, alternatively, consisting of at least one poloxamer or a mixture of poloxamers and, optionally, additives and/or excipients and/or adjuvants selected from among those capable of promoting the formation and stabilisation of the gel, selected from the group comprising or, alternatively, consisting of acid salts, sodium sorbate, potassium sorbate, sodium benzoate, potassium benzoate, glycols, ethylene glycol and propylene glycol;
wherein said composition comprises or, alternatively, consists of an effective amount of a microorganism (i) selected from the group comprising or, alternatively, consisting of the microorganisms in Table 1; preferably said at least one microorganism is selected from the group comprising or, alternatively, consisting of the strains: Lactobacillus saliverius (LS01) DSM 22775, deposited on 23 Jul. 2009; Bifidobacterium breve (BR03) DSM 16604, deposited on 20 Jul. 2004; Lactobacillus pentosus (LPS01) DSM 21980, deposited on 14 Nov. 2008; Streptococcus thermophilus (FP4) DSM 18616, deposited on 13 Sep. 2006; Lactobacillus casei ssp. rhamnosus (LR04) DSM 16605, deposited on 20 Jul. 2004; and Lactobacillus acidophilus (LA02) DSM 21717, deposited on 6 Aug. 2008; even more preferably the strain is Lactobacillus salivarius (LS01) DSM 22775, and/or Bifidobacterium breve (BR03) DSM 16604, and/or L. pentosus (LPS01) DSM 21980, and
wherein said composition is for topical transdermal use in the preventive and/or curative treatment of pathologies, disorders or diseases associated with/deriving from alterations of the immune system selected from the group comprising allergies, atopy, allergic rhinitis, food hypersensitivity, dermatitis, atopic dermatitis, eczema, psoriasis, asthma and immunodeficiencies.

2. The composition for use according to claim 1, wherein said poloxamer is selected from the group comprising or, alternatively, consisting of Poloxamer 124 CAS N. 9003-11-6 with an average molecular weight of 2090-2360, Poloxamer 188 (Lutrol® F66) CAS No. 9003-11-6 with an average molecular weight of 7680-9510, Poloxamer 237 with an average molecular weight of 6840-8830, Poloxamer 338 with an average molecular weight of 12700-17400, Poloxamer 407 (Lutrol® F127 Prill) CAS No. 9003-11-6 with an average molecular weight of 9840-14600, or mixtures thereof; preferably said Poloxamer 188 is present in an amount comprised from 0.1% to 10% by weight, out of 100 g of composition, preferably in an amount comprised from 0.5% to 5% by weight, for example from 1% to 3% by weight; and/or preferably said Poloxamer 407 is present in an amount comprised from 1% to 40% by weight, out of 100 g of composition, preferably in an amount comprised from 5% to 30% by weight, for example from 10% to 20% by weight.

3. The composition for use in accordance with one of claims 1-2, further comprising, as an active substance (iii), an effective amount of at least one compound selected from the group comprising or, alternatively, consisting of a flavonoid such as rutin, rutoxides, arbutin, oxerutin, diosmin, hesperidin and troxerutin, chemotherapeutic agents, arctigenin, arctin, berberine, berbamine, sanguinarine and chelerythrine, as such or in the form of plant extracts containing said compounds, on their own or in combination with one or more chemotherapeutic agents or anti-tumour drugs.

4. The composition for use in accordance with any one of the preceding claims, wherein:
said microorganisms are present at a concentration comprised from 1×10⁶ CFU/g to 1×10¹² CFU/g of composition, preferably from 1×10⁷ CFU/g to 1×10¹¹ CFU/g of composition, even more preferably from 1×10⁸ CFU/g to 1×10¹⁰ CFU/g of composition, for example 1×10⁹ CFU/g of composition, and wherein:
said microorganisms are present in said composition in an amount by weight comprised from 0.1% to 5%, preferably in an amount by weight comprised from 0.5% to 3%, even more preferably in an amount by weight comprised from 1% to 2%, relative to the total weight of composition.

5. The composition for use in accordance with claim 1 or 2, wherein:
said composition comprises an effective amount of an active substance selected from:
(iii) at least one compound selected from the group comprising or, alternatively, consisting of a flavonoid such as rutin, rutoxides, arbutin, oxerutin, diosmin, hesperidin and troxerutin, chemotherapeutic agents, arctigenin, arctin, berberine, berbamine, sanguinarine and chelerythrine, as such or in the form of plant extracts containing said compounds, on their own or in combination with one or more chemotherapeutic agents or anti-tumour drugs; and/or
(ii) melatonin and/or the natural and/or synthetic derivatives thereof;
said active substance being present in said composition in an amount by weight comprised from 0.1% to 10% by weight, preferably in an amount comprised from 0.5% to 5% by weight, even more preferably from 1% to 3% by weight, relative to the total weight of the composition.

6. The composition in accordance with any one of claims 1-5, for use:
in the treatment of pathologies, disorders or diseases due to the pathological malfunctioning of physiological functions connected with circadian rhythms;
in the treatment of pathologies, disorders or diseases due to an abnormal or excessively high or excessively low blood pressure;
in the treatment of pathologies, disorders or diseases due to an excess of free radicals and oxidative stress;
in the treatment of pathologies, disorders or diseases due to an imbalance of the immune system;
in the treatment of pathologies, disorders or diseases due to inflammatory states;
in the treatment of pathologies, disorders or diseases due to acquired immunodeficiency;
in the treatment of pathologies, disorders or diseases due to viral infections and bacterial infections;
in a treatment as a support or adjuvant to chemotherapy;
in the treatment of cancer with chemotherapeutic agents;
in the treatment of sleep disorders in order to favour, in the treated subjects, the quality of sleep and regularity of sleep, with particular reference to REM sleep;
in the treatment of pathologies, disorders or diseases of the eyes, of the retina, of the iris, of the optic nerve, retinal detachment and macular degeneration;
in the treatment of vision disorders, in particular in maculopathy of the retina, via external applications in the vicinity of the eye and the temple;
in the treatment of pathologies, disorders or diseases due to autism and Down's syndrome, in particular in children and adolescents, by favouring balance and control also of states of anxiety;
in the treatment of pathologies, disorders or diseases due to senile dementia or Alzheimer's disease;
in the treatment of pathologies, disorders or diseases due to Parkinson's disease or neurological dysfunctions, by improving sleep and tremors;
in the treatment of pathologies, disorders or diseases due to pulmonary cystic fibrosis in children, by performing an anti-inflammatory activity;
in the treatment of prostate tumours, by preventing the propagation thereof;
in the treatment of tumours, as an anti-inflammatory, anti-tumour adjuvant;
in the treatment of pathologies, disorders or diseases due to heart attack, myocardial infarction, cardiopathies, cardiac or coronary failure or myocardial insufficiency.

7. A process for the preparation of a composition in accordance with any one of the preceding claims, said process comprising the following steps:
mixing in water, under continuous stirring, the various components, added in succession one after the other or separately premixed and added together, using a mixer provided with a temperature control and adjustment means and a stirring means, wherein the temperature is set at values lower than room temperature, comprised from 2° C. to 16° C., preferably from 4° C. to 10° C., for example from 6° C. to 8° C., for a time comprised from 1 minute to 15 minutes so as to obtain a homogeneous mixture;
maintaining said homogeneous mixture, preferably under continuous stirring, at a temperature comprised from 2° C. to 10° C., preferably at a temperature comprised from 4° C. to 8° C., for example from 5° C. to 7° C., for a time comprised from 1 minute to 60 minutes, preferably from 5 minutes to 40 minutes, even more preferably from 10 minutes to 20 minutes, so as to obtain said composition in a liquid state;
packaging said composition in sealed containers.
